(19) United States
(12) Patent Application Publication
(10) Pub. No.: US 2021/0290915 A1
(43) Pub. Date: Sep. 23, 2021

(54) GUIDE WIRE

(71) Applicant: YOKOWO CO., LTD., Tokyo (JP)

(72) Inventors: Koichi SAKAI, Tokyo (JP); Naoki OOKA, Tomioka-Shi, Gunma (JP)

(21) Appl. No.: 17/263,820

(22) PCT Filed: Mar. 6, 2019
(86) PCT No.: PCT/JP2019/008779

§ 371 Date: Jan. 27, 2021

(30) Foreign Application Priority Data
Aug. 8, 2018 (JP) 2018-148978

(51) Int. Cl. A61M 25/09; A61M 25/00

(52) U.S. Cl. CPC A61M 25/09; A61M 2025/09133; A61M 2025/09083; A61M 25/0045

(57) ABSTRACT

The present invention provides a guide wire that has a distal end portion having excellent flexibility and sufficient strength and has excellent torque transmission performance. A guide wire 10 has a core wire 2, a torque tube 4 having a base end joined to a distal end of the core wire 2, and an insertion distal end portion 6 joined to a distal end of the torque tube 4, and may further have a helical body 8 disposed outside the torque tube 4 and having a distal end joined to the insertion distal end portion 6, in which the torque tube 4 is a hollow member including a tubular constituent layer 11 formed in such a way that a plurality of wires spirally wound in a same direction is combined in parallel.

### TECHNICAL FIELD

The present invention relates to a guide wire to be used in a medical field.

### BACKGROUND ART

A guide wire is used as a medical tool that is used as a guide when a catheter is inserted into a digestive organ, a blood vessel, a ureter, or the like. A general guide wire for medical use is provided with a core wire having a flexibility (softness) and a coil body arranged at the distal end of the core wire. By joining the distal end of the core wire and the distal end of the coil body, a tip to be an insertion distal end portion into a body is formed.

In such a guide wire for medical use, it is required that the distal end be soft and have excellent flexibility, turning force at hand be transmitted sufficiently to the distal end, in other words, so-called torque transmission performance be excellent, etc. To meet these requirements, for example, a guide wire provided with a core wire of which a portion is covered with a mesh-like tube portion (blade tube) formed by weaving filaments composed of stainless steel or the like, the tube portion composed of a super-elastic alloy, has been proposed (Patent Literature 1). In addition, a guide wire provided with a coil and a linear core material disposed in such a way as to penetrate the coil and reach to an insertion distal end has been proposed (Patent Literature 2).

### CITATION LIST

### Patent Literature

Patent Literature 1: Japanese Patent No. 3135115

Patent Literature 2: Japanese Patent No. 5806441

### SUMMARY OF INVENTION

### Technical Problem

The guide wires proposed in Patent Literatures 1 and 2 have satisfactory torque transmission performance; however, it cannot necessarily be said that the flexibility is sufficient. Making a core wire rigid in order to improve the torque transmission performance makes the flexibility liable to be impaired. On the other hand, using a soft core wire in order to improve the flexibility makes it difficult to improve the torque transmission performance and makes the strength liable to be lowered. That is, the torque transmission performance and the flexibility are in a so-called trade-off relationship, and therefore it has been difficult to achieve both of these characteristics.

The present invention has been completed in view of such circumstances, and an object of the present invention is to provide a guide wire that has a distal end portion having excellent flexibility and sufficient strength and has excellent torque transmission performance.

### Solution to Problem

The first aspect of the present invention is a guide wire provided with: a core wire; a torque tube having a base end joined to a distal end of the core wire; and an insertion distal end portion joined to a distal end of the torque tube; wherein the torque tube is a hollow member including a tubular constituent layer formed in such a way that a plurality of wires spirally wound in a same direction is combined in parallel.

The second aspect of the present invention is the guide wire according to the first aspect, further having a helical body disposed outside the torque tube, wherein a distal end of the helical body is joined to the insertion distal end portion.

The third aspect of the present invention is the guide wire according the second aspect, wherein the helical body is a tapered helical body having a diameter gradually reduced toward the distal end of the helical body.

The fourth aspect of the present invention is the guide wire according to the first to the third aspect, wherein the torque tube has a multilayered structure such that two or more tubular constituent layers are disposed in such a way as to be stacked in a radial direction, and winding directions of the respective wires constituting the tubular constituent layers adjacently disposed are mutually opposite directions.

The fifth aspect of the present invention is the guide wire according to the second to the third aspect, wherein a winding direction of a wire constituting the helical body and a winding direction of a plurality of the wires constituting the torque tube are mutually opposite directions.

### Advantageous Effects of Invention

According to the above-described aspects of the present invention, it is possible to provide a guide wire that has a distal end portion having excellent flexibility and sufficient strength and has excellent torque transmission performance.

### BRIEF DESCRIPTION OF DRAWINGS

FIG. 1 is a side view schematically showing the first embodiment of a guide wire of the present invention.

FIG. 2 is a sectional view schematically showing a vicinity of a distal end of the guide wire in FIG. 1.

FIG. 3 is a perspective view showing one example of a torque tube.

FIG. 4 is a sectional view schematically showing a vicinity of a distal end of the second embodiment of a guide wire of the present invention.

FIG. 5 is a perspective view showing another example of a torque tube.

FIG. 6 is a side view schematically showing the third embodiment of a guide wire of the present invention.

FIG. 7 is a sectional view schematically showing a vicinity of a distal end of the guide wire in FIG. 6.

FIG. 8 is a side view schematically showing the fourth embodiment of a guide wire of the present invention.

FIG. 9 is a sectional view schematically showing a vicinity of a distal end of the guide wire in FIG. 8.

### DESCRIPTION OF EMBODIMENTS

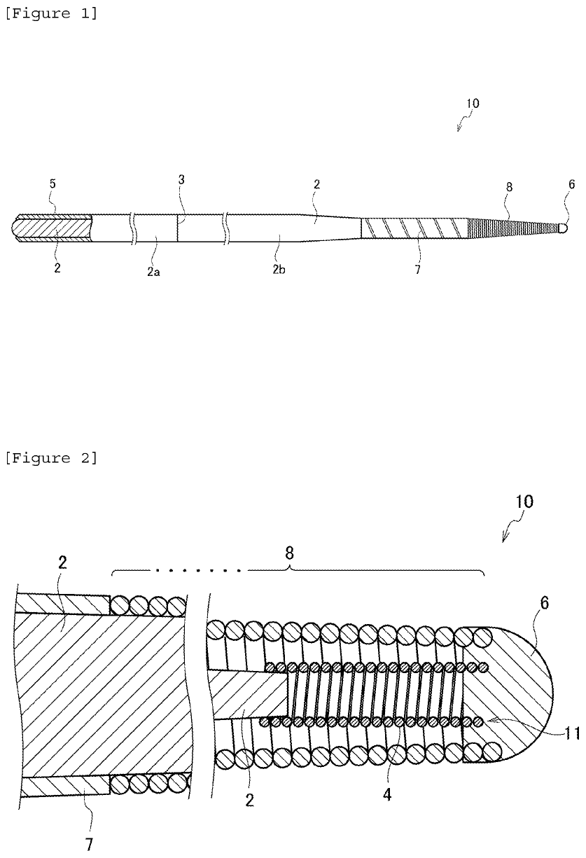

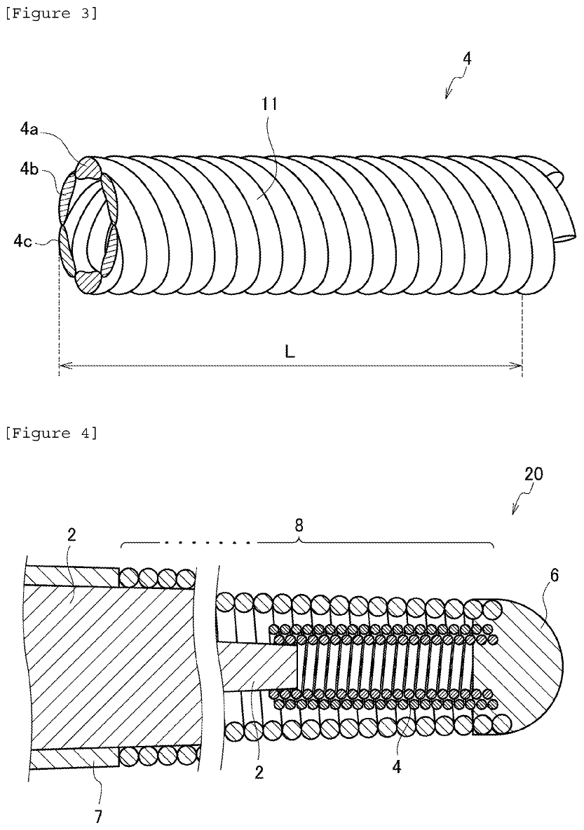

Hereinafter, embodiments of the present invention will be described, but the present invention is not limited to the following embodiments. FIG. 1 is a side view schematically showing the first embodiment of a guide wire of the present invention. FIG. 2 is a sectional view schematically showing a vicinity of a distal end of the guide wire in FIG. 1. As shown in FIG. 1 and FIG. 2, the guide wire 10 of the present embodiment is provided with: a core wire 2; a torque tube 4 having a base end joined to a distal end of the core wire 2; and an insertion distal end portion 6 joined to a distal end of the torque tube 4. The distal end of the core wire 2 is joined at a position where the distal end of the core wire 2 enters an inside of the torque tube 4 to, for example, about 1 to about 3 mm. In addition, a helical body 8 having a distal end joined to the insertion distal end portion 6 is disposed outside the torque tube 4.

FIG. 3 is a perspective view showing one example of the torque tube. As shown in FIG. 3, the torque tube 4 which is used as a constituent member for the guide wire 10 (FIG. 1 and FIG. 2) is a hollow member and is constituted by a tubular constituent layer 11 formed in such a way that a plurality of wires 4a, 4b, 4c, . . . , spirally wound in a same direction is combined in parallel. A conventional coil body is disposed at the distal end portion of the guide wire and is constituted by spirally winding one wire. When the softness of the torque tube 4 and the softness of the conventional coil body are compared, the torque tube 4 including the tubular constituent layer 11 having such constitution exhibits softness that is equal to or higher than the softness of the coil body. In addition, the torque tube 4 is more durable and has more sufficient strength as compared to the conventional coil body, and has more excellent torque transmission performance than the conventional coil body. Further, unlike the mesh-like tube portion (blade tube) proposed in Patent Literature 1, which is used for a guide wire and is formed by weaving filaments, the torque tube 4 is softer and more easily bendable, and has more excellent flexibility because a plurality of wires, 4a, 4b, 4c, . . . , is not woven (not mutually crossed).

Examples of the shape of the section of the wires which constitute the guide wire include a circular shape and an elliptical shape. It is preferable to use the torque tube constituted by a plurality of wires of which the sections have a circular shape because the guide wire having more improved softness is obtained. It is preferable to use the torque tube constituted by wires of which the sections have an elliptical shape because the guide wire having more improved torque transmission performance is obtained.

As shown in FIG. 2, the distal end of the core wire 2 is joined to the base end of the torque tube 4 and does not reach to the insertion distal end portion 6. Therefore, the softness of the torque tube 4 is exhibited effectively and the distal end portion of the guide wire 10 exhibits excellent flexibility. Thus, the distal end portion of the guide wire 10 bends softly along the shape of a narrow part without imposing a burden on tissues and the like in the body. Further, as described above, the torque tube 4 is more durable as compared to a conventional coil body. Therefore, the distal end portion of the guide wire 10 of the present embodiment has sufficient strength and has excellent torque transmission performance even when the constitution is such that the core wire 2 does not penetrate the inside (hollow part) of the torque tube 4, and the distal end of the core wire 2 is joined to the base end of the torque tube 4.

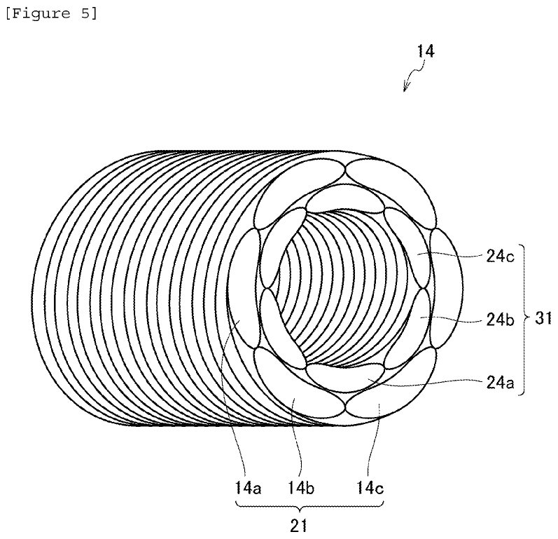

FIG. 4 is a sectional view schematically showing the vicinity of the distal end of the second embodiment of the guide wire of the present invention. FIG. 5 is a perspective view showing another example of the torque tube. As shown in FIG. 4 and FIG. 5, the torque tube 14 has a multilayered structure such that two or more tubular constituent layers 21, 31 are disposed in such a way as to be stacked in the radial direction. Further, it is preferable that winding directions of respective wires 14a, 14b, 14c, . . . , and 24a, 24b, 24c, . . . , constituting the tubular constituent layers 21, 31 adjacently disposed are mutually opposite directions. By adjacently disposing the tubular constituent layers such that the winding directions of the wires are in a relationship of mutually opposite directions, the guide wire having excellent torque transmission performance to both of the directions of rotation (clockwise/anticlockwise) can be made. The numbers of wires constituting the respective tubular constituent layers adjacently disposed may be the same with or different from each other. The diameters of the wires constituting the respective tubular constituent layers adjacently disposed may also be the same with or different from each other.

It is preferable that the number of wires per tubular constituent layer constituting the torque tube be 2 or more, more preferably 3 or more, and particularly preferably 4 or more. By setting the number of wires to be used to the above-described range, the torque tube which is sufficiently durable and has more excellent torque transmission performance can be made. The upper limit of the number of wires is not particularly limited, but is preferably set to 20 or less, more preferably 14 or less, and particularly preferably 8 or less. When the number of the wires is too large, the softness of the torque tube is liable to be lowered, and production is liable to be difficult.

The total length L (FIG. 3) of the torque tube 4 is usually 3 to 100 mm, preferably 4 to 90 mm, and more preferably 5 to 80 mm. The total length of the torque tube can appropriately be set according to the intended use and the like of the guide wire.

The outer diameter and inner diameter of the torque tube each are slightly different depending on the layer structure (single-layered structure/multi-layered structure) of the torque tube, the number of wires constituting the torque tube, and the like. For example, the inner diameter (maximum inner diameter) of the torque tube having a number of wires of 6 and having a single-layered structure is usually 0.076 to 0.813 mm, and preferably 0.102 to 0.559 mm. The outer diameter (maximum outer diameter) of the torque tube having a number of wires of 6 and having a single-layered structure is 0.152 to 0.889 mm, and preferably 0.178 to 0.635 mm.

The inner diameter (maximum inner diameter) of the torque tube having a number of wires of 6 and having a two-layered structure is usually 0.114 to 0.813 mm, and preferably 0.140 to 0.559 mm. The outer diameter (maximum outer diameter) of the torque tube having a number of wires of 6 and having a two-layered structure is usually 0.267 mm to 0.889 mm, and preferably 0.292 to 0.635 mm.

The wires constituting the torque tube are formed with a metal material. Examples of the metal material include: stainless steel, such as SUS302, SUS304V, and SUS316L; and various alloys, such as a nickel-titanium alloy (such as, for example, Nitinol), and Co, Cr, W and platinum alloys. Among others, stainless steel, such as SUS304V, is preferable. The diameter (maximum diameter) of the wires may be, for example, about 0.025 to about 0.152 mm.

The total length and outer diameter of the core wire constituting the guide wire can appropriately be set according to the intended use (for digestive organs, for blood vessels, etc.) of the guide wire. The total length of the core wire is, for example, 800 to 5,500 mm, and preferably 1,300 to 4,800 mm. The outer diameter of the core wire may gradually be reduced from the base end (at hand) toward the distal end. The outer diameter of the core wire is, for example, 0.254 to 0.889 mm, and preferably 0.305 to 0.711 mm.

The core wire is formed with a metal material having satisfactory flexibility. Examples of the metal material include: stainless steel, such as SUS302, SUS304V, and SUS316L; and various alloys, such as a nickel-titanium alloy (such as, for example, Nitinol), and Co, Cr, and W alloys. Among others, stainless steel, such as SUS304V, and a nickel-titanium alloy, such as Nitinol, are preferable, and from the viewpoint of having excellent kink resistance, and the like, a nickel-titanium alloy, such as Nitinol, is particularly preferable.

As shown in FIG. 1, it is preferable to use the core wire 2 obtained in such a way that a base end side 2a and a distal end side 2b are constituted by different metal materials, and these are joined at a joint 3. The stainless steel is a metal material that cannot be said to have satisfactory kink resistance but has excellent torque transmission performance. The nickel-titanium alloy is a metal material that is liable to bring about torque loss but has excellent kink resistance. For example, when the core wire 2 obtained in such a way that the base end side 2a where turning force is generated is constituted by stainless steel, and the distal end side 2b which penetrates into an operation site which is more bent is constituted by a nickel-titanium alloy is used, both of excellent torque transmission performance and kink resistance can thereby be achieved. It is preferable to make the length of the distal end side 2b constituted by a nickel-titanium alloy or the like 300 mm or longer.

The helical body is formed with a metal material having satisfactory flexibility. Examples of the metal material include: stainless steel, such as SUS302, SUS304V, and SUS316L; and various alloys, such as a nickel-titanium alloy (such as, for example, Nitinol), a platinum alloy, and gold and W alloys. Among others, it is preferable to constitute the helical body by a metal material, such as a platinum alloy, which is less likely to transmit an X-ray. By adopting such constitution, X-ray photography or the like can be performed with a high contrast, the position or the like at an operation site can be grasped accurately and easily.

The total length of the helical body is usually 10 to 800 mm, and preferably 20 to 600 mm. The total length of the helical body can appropriately be set according to the intended use and the like of the guide wire. The diameter (maximum diameter) of the wire constituting the helical body may be, for example, about 0.040 to about 0.080 mm. The wire constituting the helical body may be wound closely or may be wound sparsely.

As shown in FIG. 1 and FIG. 2, the helical body 8 preferably has a tapered shape of which the diameter gradually reduces toward the distal end of the helical body 8. By using such a tapered helical body 8, the guide wire which is easily inserted into a narrower vessel or the like and in which the distal end having a thinner diameter is softer can be made. In addition, it is preferable that the winding direction of the wire constituting the helical body 8 and the winding direction of a plurality of the wires, 4a, 4b, 4c, . . . , (FIG. 3), constituting the torque tube 4 be mutually opposite directions. Thereby, the guide wire having excellent torque transmission performance to both of the directions of rotation (clockwise/anticlockwise) can be made.

A covering layer 5 is formed on a surface of the core wire 2 (FIG. 1). By forming the covering layer 5, the sliding resistance of the guide wire can be reduced, and the operability can be improved. The material for forming the covering layer 5 is preferably a hydrophobic resin material. Among others, it is preferable to form the covering layer with a fluorine-based resin, such as PTFE, ETFE, and PFA. By forming the covering layer in this way, the sliding resistance of the guide wire can be reduced more effectively.

A visible marker (marker 7) having a spiral pattern or the like is given in the vicinity of the distal end of the core wire 2 in order to enhance the visibility by a fiber scope or the like at an operation site (FIG. 1). Such a marker 7 can be given in such a way that, for example, a predetermined portion of the core wire 2 is covered with a heat-shrinkable resin tube to which an appropriate pattern (marker) is given, and the heat-shrinkable resin tube is then heated and, if necessary, fixed with a UV adhesive or the like, or by other methods.

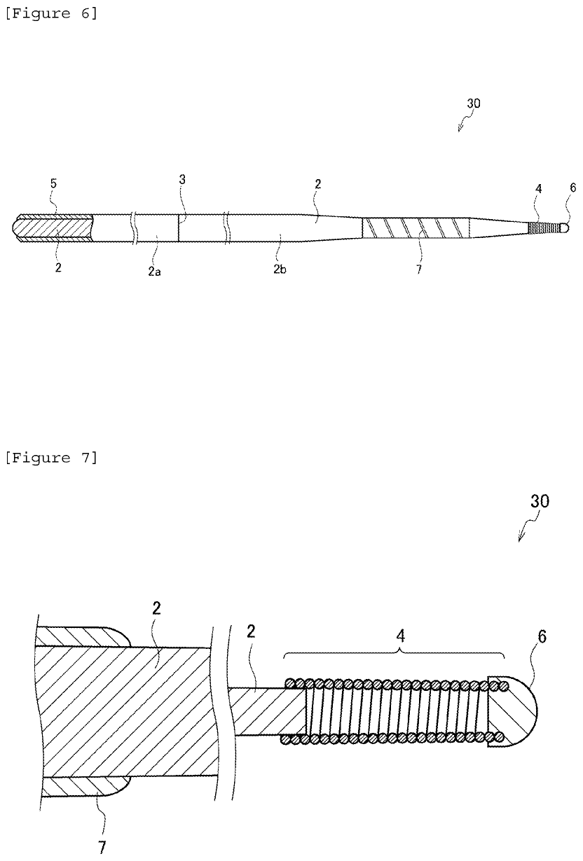

FIG. 6 is a side view schematically showing the third embodiment of the guide wire of the present invention. FIG. 7 is a sectional view schematically showing the vicinity of the distal end of the guide wire in FIG. 6. A guide wire 30 of the embodiment shown in FIG. 6 and FIG. 7 is provided with: a core wire 2; a torque tube 4 having a base end joined to a distal end of the core wire 2; and an insertion distal end portion 6 joined to a distal end of the torque tube 4. Unlike the guide wire 10 of the embodiment shown in FIG. 1 and FIG. 2, in the guide wire 30 of the present embodiment, a helical body is not disposed outside the torque tube 4. In this way, the helical body is a constituent which can appropriately be installed according to the intended use and the like of the guide wire.

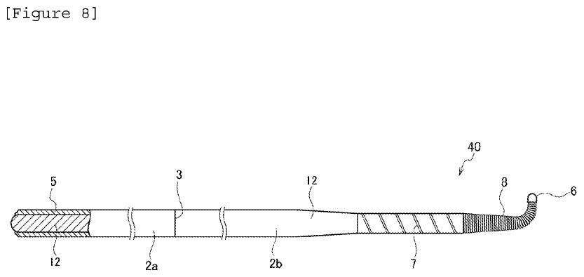

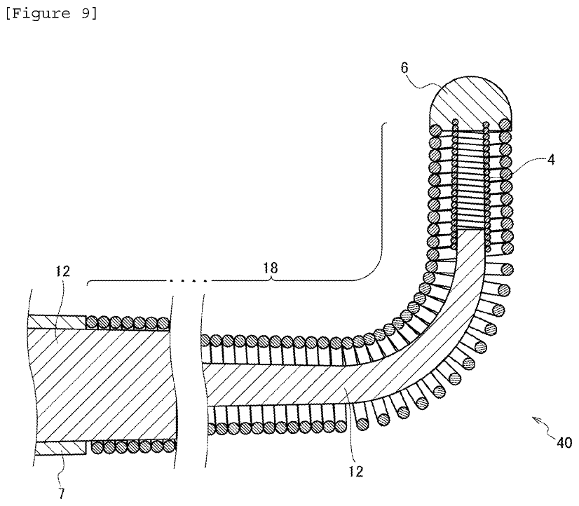

FIG. 8 is a side view schematically showing the fourth embodiment of the guide wire of the present invention. FIG. 9 is a sectional view schematically showing the vicinity of the distal end of the guide wire in FIG. 8. A guide wire 40 of the embodiment shown in FIG. 8 and FIG. 9 is provided with: a core wire 12 having a bent distal end; a torque tube 4 having a base end joined to the distal end of the core wire 12; and an insertion distal end portion 6 joined to a distal end of the torque tube 4. A helical body 18 having a bent distal end joined to the insertion distal end portion 6 is disposed outside the torque tube 4. That is, the guide wire 40 of the embodiment shown in FIG. 8 and FIG. 9 is a guide wire of an angle type having a bent distal end. In this way, the distal end of the guide wire may appropriately be bent according to the intended use and the like.

The guide wire of the present invention can be produced in accordance with a conventionally known method, except that a particular torque tube is used. For example, a core wire having a desired shape is obtained by polishing a wire made of a nickel-titanium alloy, such as Nitinol. After the obtained core wire is covered with a hydrophobic resin material, such as a fluorine-based resin such as PTFE, to form a covering layer, a predetermined portion is covered with a heat-shrinkable resin tube to which a visible marker having a spiral pattern is given. Subsequently, the base end of the torque tube is joined to the distal end of the core wire with a solder joint or the like in a state where the distal end of the core wire is allowed to penetrate inside the torque tube by about 1 to about 3 mm. Further, the insertion distal end portion is formed by joining the base end of the helical body made of a metal material, such as a platinum alloy, to the core wire with a solder joint or the like, and joining the distal end of the torque tube and the distal end of the helical body by Tig welding or the like. Thereafter, both of the opening ends of the heat-shrinkable resin tube are fixed to the core wire using a UV-curable adhesive or the like, and heating is performed to shrink the resin tube, thereby allowing the resin tube to adhere closely to the core wire. Further, if necessary, the portion or the like where the helical body is disposed is subjected to hydrophilic coating, and the guide wire of the present invention can thereby be obtained.

When the guide wire of an angle type having a bent distal end, as shown in FIG. 8 and FIG. 9, is produced using a torque tube having a relatively long total length (for example, around 70 mm), the torque tube is usually bent and fixed. Specifically, the torque tube is bent to a desired angle, and the bent portion can be fixed by adhesion with a UV adhesive, soldering, or the like. By bending the torque tube in this way, the guide wire of an angle type having a bent distal end can be produced even when a relatively long torque tube is used.

### INDUSTRIAL APPLICABILITY

The guide wire of the present invention is useful as, for example, a guide wire for a digestive organ.

### REFERENCE SIGNS LIST

2, 12: Core wire
2a: Base end side
2b: Distal end side
3: Joint
4, 14: Torque tube
4a, 4b, 4c, 14a, 14b, 14c, 24a, 24b, 24c: Wire
5: Covering layer
6: Insertion distal end portion
7: Marker
8, 18: Helical body
10, 20, 30, 40: Guide wire
11, 21, 31: Tubular constituent layer

1. A guide wire comprising:
a core wire;
a torque tube having a base end joined to a distal end of the core wire; and
an insertion distal end portion joined to a distal end of the torque tube; wherein
the torque tube is a hollow member including a tubular constituent layer formed in such a way that a plurality of wires spirally wound in a same direction is combined in parallel.

2. The guide wire according to claim 1, further comprising a helical body disposed outside the torque tube, wherein
a distal end of the helical body is joined to the insertion distal end portion.

3. The guide wire according to claim 2, wherein the helical body is a tapered helical body having a diameter gradually reduced toward the distal end of the helical body.

4. The guide wire according to claim 1, wherein
the torque tube has a multilayered structure such that two or more tubular constituent layers are disposed in such a way as to be stacked in a radial direction, and
winding directions of the respective wires constituting the tubular constituent layers adjacently disposed are mutually opposite directions.

5. The guide wire according to claim 2, wherein a winding direction of a wire constituting the helical body and a winding direction of a plurality of the wires constituting the torque tube are mutually opposite directions.
